(19) United States
(12) Patent Application Publication
(10) Pub. No.: US 2014/0058425 A1
(43) Pub. Date: Feb. 27, 2014

(54) MANUALLY OPERATED SURGICAL DEVICES WITH OPERATIVE PORTIONS FORMED OF A SEE-THROUGH MATERIAL

(71) Applicant: Amir Porat, Yehud (IL)

(72) Inventor: Amir Porat, Yehud (IL)

(21) Appl. No.: 13/594,931

(22) Filed: Aug. 27, 2012

(51) Int. Cl. A61B 17/28; A61B 17/32; A61B 17/34; A61B 17/00

(52) U.S. Cl. 606/167; 606/1; 606/205; 606/208

(57) ABSTRACT

The disclosure is directed to a manually operated surgical device comprises a proximal control means portion for manually operating the device; and a see-through distal operative means portion operably coupled to the proximal control means, wherein the distal operative means portion is formed of a material that is different than the material forming the proximal control means portion.

### BACKGROUND

The disclosure generally relates to manually operated surgical tools. Specifically, the disclosure relates to disposable manually operated surgical tools having operational portions that are comprise see-through material different than the device control portion.

Prior to World War 2, the majority of manually operated surgical instruments used in hospitals were non-disposable and/or re-usable rendered so by sterilization and/or disinfection, with the help of relatively cheap labor.

During the 50's and the 60's, as a result of the development of engineered thermoplastic materials (ETP's, enabling reduction in the cost of the devices) and following the technological improvements in manufacturing processes and material engineering, a substantial reduction of disposable instruments' costs became possible making some disposable tools competitive with the re-usable ones.

In recent years, the shift from re-usable to disposable instruments, in both the medical sector and the private sector became a priority due to the high risk of cross-infection and contamination in hospitals, clinics, and at home. However, in certain manually operated surgical devices, the portion in contact with the patient's tissue (thus the potential for infection and the need for refinement) mass production techniques may present a problem because the quality of the disposable instruments is not satisfactory and precise enough to handle and be used in especially delicate procedures and often has different physical and structural constraints that require different processing methodologies.

Accordingly, there is a need for disposable, manually operated surgical devices with operational portions that is separate and distinct from the control portions

### SUMMARY

Disclosed, in various embodiments, are monolithic, disposable manually operated surgical tools having operational portions that are separate and distinct from the control portions.

In an embodiment, a monolithic, manually operated surgical device comprises a thermoplastic proximal control means portion for manually operating the device; and a distal operative means portion, operably coupled to the proximal control means, wherein the distal operative means portion is of different material than the thermoplastic proximal control means portion.

In another embodiment, the distal operative means portion for operating the devices described, is see-through.

In yet another embodiment of the devices provided herein, the distal operative means portion for operating the devices described is configured to form a magnifying lens.

In an embodiment of the devices provided herein, the distal operative means portion for operating see-through devices described is configured to avoid obstructing the operator vision by allowing the incident light to go throw, and, not block user's view on the treatment area.

These and other features of the manually operated surgical tools having a distal operative means portion that is comprised of a material that is different than a proximal control means portion will become apparent from the following detailed description when read in conjunction with the drawings, which are exemplary, not limiting, and wherein like elements are numbered alike in several figures.

### BRIEF DESCRIPTION OF THE FIGURES

For a better understanding of the disposable, manually operated surgical tools having a distal operative means portion that is comprised of a material that is different than a proximal control means portion, with regard to the embodiments thereof, reference is made to the accompanying drawings, in which like numerals designate corresponding elements or sections throughout and in which:

FIG. 1 shows forceps in accordance with an embodiment of the manually operated surgical device;

FIG. 2 shows an ophthalmic forceps, used also as a suture tying and corneal forceps in accordance with an embodiment of the manually operated surgical device;

FIG. 3 shows a magnification of the see-through material portion holding a tissue which allow and enable the user to see the tissue in accordance with an embodiment of the manually operated surgical device of FIG. 2;

FIG. 4 shows dressings forceps in accordance with an embodiment of the manually operated surgical device;

FIG. 5 shows artery hemostat forceps in accordance with an embodiment of the manually operated surgical device;

FIG. 6, shows the distal operative means portion for operating the device described in FIG. 3, made with a see-through material;

FIG. 7, shows the distal operative means portion for operating the device described in FIGS. 1 and 2; and

FIG. 8, shows a manipulating ophthalmic needle hook in accordance with an embodiment of the manually operated surgical device.

### DESCRIPTION

Provided herein are disposable, manually operated surgical tools having distal operational means portions that are separate and distinct from the control portions. By providing these portions in an different materials, certain advantages can be derived, such as by reducing the size of the portions requiring precise manufacturing and that have low variability tolerance, thereby reducing costs, and manufacturing complexity, while maintaining hygienic integrity of the device. The device can be single-use sterile disposable devices, and sterilizable. The entire device can also be reusable and sterilizable device.

The distal control means portions can be made with metal or regular thermoplastic materials and the tips can be made of, for example, an injection molded strong expensive see-through thermoplastic material, which can be reinforced with, for example, glass or metal fiber, graphene nanobodies, polyamic acid, carbon nanotubes, a reinforcing solvent, or a combination comprising at least one of the foregoing, and there is no need to form the whole instrument (in other words, the whole device) out of expensive hard to inject mold parts. The only part used and needed to be very precise can be made out of a stiffer ETP, adapted to injection molding. The distal operational means portion can also be opaque.

The term “reusable” refers to a portion of the surgical device for use by a care provider in performing a procedure which can be sterilized and reused in subsequent procedures and/or that can be utilized several times and maintains similar quality as when used the first time.

The term “disposable” when applied to a component, is a broad term and means, without limitation, that the component in question is used for the first time, or a finite number of times for the same patient or user and then discarded. Some disposable components can be used only once and then discarded. Other disposable components can be used more than once on the same procedure and then discarded. In some embodiments, the manually operated surgical device is a single-use component. Such portions can assure that a sterile component is contacting a surgical site and is free of any contamination.

The monolithic (in other words, a structure having, or acting, as a single, uniform structure), disposable manually operated surgical devices disclosed can, for example, be surgical forceps or also called hemostat. Forceps are commonly held between the thumb and two or three fingers of one hand, with the top end resting on the anatomical snuff at the base of the thumb and index finger or, alternatively have 2 rings on each side to put the fingers in. Spring tension (in other words, biasing means) at the proximal end can hold the grasping ends apart until pressure is applied. This allows one to quickly and easily grasp small objects or tissue to move and release it or to grasp and hold tissue with easily variable pressure. Thumb forceps can be used to hold tissue in place when applying sutures, to gently move tissues out of the way or block veins during exploratory surgery and to move dressings or draping without using the hands or fingers. Forceps can have smooth tips, cross-hatched tips or serrated tips (often called ‘mouse's teeth’). Common arrangements of teeth are 1×2 (two teeth on one side meshing with a single tooth on the other), 7×7 and 9×9. Serrated forceps are used on tissue; counter-intuitively, serrated tips may damage tissue less than a smooth surface (grasping is done with less overall pressure). Smooth or cross-hatched forceps are used to move dressings, remove sutures and similar tasks. The forceps can have handles and also used as a needle holder or Mosquito hemostat (in other words, mosquito artery forceps), to stop blood and many other functions.

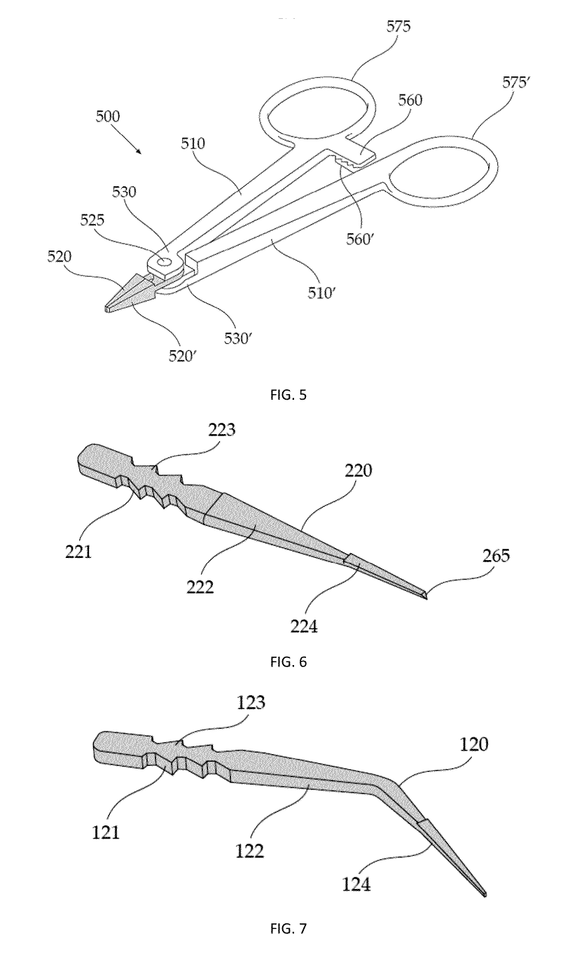

Similarly, locking forceps, sometimes called clamps, are used to grasp and hold objects or tissue. When used to compress an artery to forestall bleeding, locking forceps can also be called hemostats. Another form of locking forceps is the needle holder, used to guide a suturing needle through tissue. Many locking forceps use finger loops to facilitate handling (see e.g., FIG. 5). The finger loops can usually be grasped by the thumb and middle or ring fingers, while the index finger can help guide the instrument. An example of the locking mechanism is a series of interlocking teeth located near and/or between the finger loops. As the handle members of the forceps comprising the finger loops are closed, the teeth on each member engage the teeth on the opposite member and keep the forceps jaws' grasping surfaces from separating. A simple shift of the fingers can be all that is needed to disengage the teeth and allow the forceps jaws' grasping ends to move apart. The forceps described herein can also be used for surgery that many of them are made today under microscope or other magnifying observation.

Currently the vast majority of the forceps and/or hemostats instruments are formed from either opaque plastic or metal. By using operative portions that can also be made of see-through material, the user can observe the tissue and analyzes it in real time, whereby in other instruments, not similarly apportioned the view of the user is substantially blocked and cannot be observed. For example a surgeon cannot see the color or the way the tissue or vein or part he may be holding looks. These attributes become more important when the user works in a group and/or under microscopes or other magnifying observation instruments. The see-through material disclosed, can also be formed as a lens, magnifying and increasing the image of the tissue held or otherwise manipulated by the forceps and hemostats instruments disclosed herein. Light can also pass through this transparent material and not shadow the target area, as can occur when using the typical instruments.

In an embodiment, handles of the devices provided, can be made by materials, for example, metal or regular plastic and the tips (in other words, the distal operative means portion) can be made of a see-through, injection molded, strong expensive performance engineered thermoplastic (ETP) reinforced with glass and/or metal fibers or metal Micro-, or Nanoparticles, thus obviating the need to form the whole device out of expensive hard to inject parts. Only those parts in contact with the patient that may be needed to be very precise will be made out of ETP, for example, clear or transparent thermoplastic material, such as poly(siloxane-carbonate).

In addition, the distal operative means portions of the device can be transparent, and may be see-through and made of clear plastic or glass or other transparent or translucent material, such that the user may be able to observe the item or tissue or area being gasped or handled, or otherwise manipulated, without the opacity of metal or opaque plastic part to prevent the care provider from looking at medical tissue or other objects. The transparent material can be formed to also act as a magnifying device so the user can better see the item grasped.

In an embodiment, provided is a monolithic, manually operated surgical device, comprising: a proximal control means portion for manually operating the device; and a distal operative means portion, operably coupled to the proximal control means, wherein the distal operative means portion is comprised of a see-through material that is different than the material of the proximal control means portion.

In an embodiment, the term “see-through” refers to an easiness with which a target can be visually recognized through the distal operative means portion and can be specified by total luminous transmittance and/or parallel luminous transmittance. “See-through” is envisioned to encompasses any characteristic that allows visual inspection through the distal operative means portion. Specifically, depending on the device a viewing window, or the entire distal operative means portion may be translucent, transparent, or entirely clear.

The term “translucent” indicates that light can pass through the distal operative means portion, but the light is diffused. It does not require that a whole surface or an article itself is transparent and portions of the article may be transparent or opaque, for example to serve a function or to form a decorative pattern. The term “translucent” as used herein would refer to a distal operative means portion made from a thermoplastic composition that transmits at least 60% in the region ranging from 250 nm to 700 nm (in other words, visible light range) with a haze of less than 40%. In one embodiment, the composition of the distal operative means portion has a transmission of at least 75%. In another embodiment, the composition of the distal operative means portion has a transmission of at least 85%. In yet another embodiment, the composition of the distal operative means portion has a haze of less than 40%, and in another embodiment, the composition of the distal operative means portion has a haze of less than 10%. In another embodiment, the composition of the distal operative means portion has a haze of less than 5%.

In an embodiment, the term “transparent” refers to a distal operative means portion made from a thermoplastic composition capable of at least 70% transmission of light. The light referred to can be, e.g., actinic light (e.g., from a laser), emitted light (e.g., from a fluorochrome), or both, or transmittance of at least 80%, more preferably at least 85%, and even more preferably at least 90%, as measured spectrophotometrically using water as a standard (100% transmittance) at 720 nm. The term “transparent” as used herein would also refer to a distal operative means portion made from a thermoplastic composition that transmits at least 70% in the region ranging from 250 nm to 700 nm with a haze of less than 10%.

The term “haze” as used herein refers to the percentage of diffused light transmitted by a material measured according to the ASTM D 1003 standard. The term “haze” refers in an embodiment to that percentage of light which in passing through deviates from the incident beam greater than 2.5 degrees on the average. “Haze” may be measured herein by a Byk Gardner haze meter (all haze values herein are measured by such a haze meter and are given as a percentage of light scattered).

The thermoplastic proximal control means, as well as the thermoplastic distal operative means portion, can comprise any thermoplastic material or combination of thermoplastic materials that can be formed into the desired shape and provide the desired properties. Exemplary materials include, but are not limited to thermoplastic materials, as well as combinations of thermoplastic materials with elastomeric materials, and/or thermoset materials. Possible thermoplastic materials include at least one of the foregoing polybutylene terephthalate (PBT); acrylonitrile-butadiene-styrene (ABS); polycarbonate; polycarbonate/PBT blends; polycarbonate/ABS blends; copolycarbonate-polyesters; acrylic-styrene-acrylonitrile (ASA); acrylonitrile-(ethylene-polypropylene diamine modified)-styrene (AES); phenylene ether resins; blends of polyphenylene ether/polyamide; polyamides; phenylene sulfide resins; polyvinyl chloride PVC; high impact polystyrene (HIPS); low/high density polyethylene (L/HDPE); polypropylene (PP); expanded polypropylene (EPP); Polyphthalamide (PPA); and thermoplastic olefins (TPO).

The desired properties for the proximal control means can be obtained with, for example, a simple thermoplastic material having Young's modulus of 0.8 to 10.0 GPa, for example, specifically 1.0 to 5.0 GPa, more specifically 1.5 to 4.0 GPa.

Additionally, the material used for the thermoplastic proximal control means can have a Poisson ratio of 0.3 to 0.5, for example, specifically 0.3 to 0.45, more specifically 0.3 to 0.35. The desired properties for the distal operative means portion can be obtained, for example, with a thermoplastic material having Young's modulus of 8.0 to 70 GPa, for example, specifically 10 to 50 GPa, more specifically 15.0 to 40 GPa.

As indicated, certain manually operated surgical devices are biased to the open position at the distal end of the proximal control means portion of the device. The function of any biasing means (e.g., any load bearing elastic object used to store and transfer mechanical energy) in manually operated surgical devices, requires the flexibility to provide displacement to the full range of desired motion while simultaneously being stiff enough to provide restoring force to the manually operated surgical devices to regain its initial spatial position. Ductile metals (e.g., stainless steel, titanium) are inherently flexible and have a Young's modulus that is high enough to give proper restoring force without reaching material plasticity (i.e. yield). Compared to metals, thermoplastic materials are less flexible and have lower Young's modulus.

In an embodiment, the term “distal” refers to that portion which is further from the user while the term “proximal” refers to that portion which is closer to the user or surgeon, or care provider.

External work done, for example by compression on such biasing means of the proximal control means portion of the manually operated surgical devices, causing its distal ends to deflect from their unstressed state, can be transformed into strain energy, referring to a form of potential energy. The strain energy in the form of elastic deformation can be recoverable in the form of mechanical work that may be used to restore the distal ends of the proximal control means to their original position. For a biased open proximal control means portion, the strain energy may be described by Equation (1):

$$U = \int \frac{{M}^{2} d y}{2 EI} \qquad ( Equ . 1 )$$

where:
U is the strain energy in Joules (J);
M is the Moment in Nm;
dy is the change in position of the distal ends of the proximal control means portion in m;
E is Young's modulus in N/m²; and

I is the angular moment of inertia in N·m² and is equal to (Wt³/12), where W is the width of the proximal control means portion and t is its thickness (in m).

Elastic materials such as those that can be used in the proximal control means portion described herein, when under uniaxial compression (assuming Cartesian coordinate system), in other words when under load only in the y direction, resulting e.g., from squeezing the proximal control means portion of the manually operated surgical device, will tend to expand in other directions (e.g., along the x and z axes). That degree of expansion is another indication of the stiffness of the thermoplastic material used in the proximal control means portion and is defined as the Poisson ratio. Accordingly, varying the thickness of the proximal control means portion and the selection of thermoplastic materials with proper Poisson ratio, may be beneficial in providing the necessary restoring action while maintaining the durability of the proximal control means portion.

To estimate the yield failure in ductile materials such as the reusable proximal control means portion described herein, and/or the disposable distal operative means portion described herein, the maximum Von Mises stress parameter provides a predictive value. Under load conditions where Von Mises stress at a particular location (e.g., at the distal ends of the proximal control means portion), is larger than the yield strength (e.g., the material's resistance threshold to rupture or plastic deformation under an applied load), the material will tend to yield at that location. Under load conditions where Von Mises stress is larger than the threshold strength of the material, the material would break at that location. Accordingly, when comparing the effectiveness of ductile materials, the lower the Von Mises stress on the material, such as the reusable proximal control means portion described herein, and/or the disposable distal operative means portion described herein, at a given load percentage, the more effective is the material in avoiding failure (e.g., better fault tolerance).

The thickness of the proximal control means portion can be fixed or variable along the span (L) of the proximal control means portion from the first distal end to the second opposite distal end. For example the thickness at each of the distal ends can be 0.8 to 4.0 mm, specifically 1.0 to 3.5 mm, more specifically 1.5 to 2.5 mm. For example, it may be beneficial to have the thickness at the proximal end, thicker than the nominal thickness at the distal ends of the proximal control means portion, wherein, for example, the thickness decreases continuously along the span of the proximal control means portion. In other words, the proximal control means portion has thickness that tapers continuously along the span of the proximal control means portion. The degree of tapering can be tuned to provide the desired restoring characteristics and deflection by defining a thickness ratio between the proximal end and the distal end. That ratio (e.g., the thickness of the proximal end over the thickness at the distal end) can be for example, 20:19 to 1:1, specifically, 11:10 to 2:1, more specifically 11:10 to 5:4.

The restoring force, affecting the reusability of the proximal control means portion, may also depend on the area of the proximal control means portion under load resulting from the compression of the surgical stapler's trigger. The span (L) of the proximal control means portion can be 4 to 300 mm for example, specifically 30 to 240 mm, more specifically 40 to 100 mm Also, the width (W) of the proximal control means portion can be 3.0 to 15 mm, for example, specifically 5.0 to 12 mm, more specifically 8.0 to 10 mm. Under certain circumstances a given ratio between the span and width of the proximal control means portion will provide the proper restoring force. That ratio can be 4:1 to 12:1, for example, specifically, 5:1 to 10:1.

The proximal control means portion, as well as the thermoplastic distal operative means portion can be manufactured utilizing various molding processes (e.g., injection molding, thermoforming, extrusion, etc.) to provide for example, a unitary piece assembly (e.g., a monolithic artery forceps). In an embodiment, the proximal control means portion can be formed of a thermoplastic material that is not the same as the thermoplastic material used to form the thermoplastic distal operative means portion; and is operably coupled to the proximal control means portion.

The term “injection molding” refers to all process flows where a plastic material is injected into a mold tool and molded. These include also known variants of injection compression-molding processes. A variant of the injection compression-molding is, for example, the so-called compression-molding where the plastic material is injected into an enlarged cavity and is compression-molded when the size of the cavity is decreased. Another variant of compression-molding is, for example, the so-called expansion molding, whereby the plastic material is injected into the opening mold tool and compressed when the mold tool closes. In general, a molding cycle in an injection molding includes a mold clamping process of combining separated molds to form a cavity, a filling process of filling a molten resin by using an injection section having a screw, a pressure-keeping process, a cooling process of cooling the molten resin, a mold unclamping process of separating the molds, and a molded-item take-out process. Of these molding processes, the filling, pressure keeping, and cooling operations performed by the injection section (injection operation) have effects on quality of the molded item or productivity. Therefore, for the injection molding device that automatically performs the molding processes as described above, it is important how to decide control conditions such as the amount of the control and the control timing of the injection operations. The term “injection operations” represents operations of the injection section during the molding processes including mold-clamping, filling, pressure-keeping, mold-unclamping and taking-out steps. The term “injection molding” also encompasses the relatively new advance of reaction injection molding, wherein a two-part semi-liquid resin blend is made to flow through a nozzle and into a mold cavity where it polymerizes as a result of a chemical reaction. Injection molding is the fastest of the thermoplastic processes, and thus is generally used for large volume applications such as automotive and consumer goods. The cycle times range between 20 and 60 seconds times the amount of cavity in the mold, e.g., in a mold having 12 cavity's with 20 second cycle, one mold will produce per hour 12×3×60=2160 parts. Injection molding also produces highly repeatable near-net shaped parts. The ability to mold around inserts, holes, and core material is another advantage. Finally, injection molding generally offer the best surface finish of any process. The skilled artisan will know whether injection molding is the best particular processing method to produce a given article according to the present invention. In one embodiment, pellets of the composition are dried in an oven over a suitable period, e.g., 12 hours at 120° C., molded in injection molding machine with a suitable melt temperature profile, e.g., 100-240-250-260-260° C., where the temperature of the mold is kept suitably for processing, e.g., at 60° C. “Insert molding” refers to a method of permanent mechanical bonding, which method involves the placing of a substrate in a mold and covering all or part of the inserted substrate with a second liquid or molten plastic. Care must be taken to ensure that the inserted substrate does not shift out of its intended position during the injection of high viscosity polymer melts. As used herein, the expressions “in-mold decorating”, “in-mold labeling”, and the like, refer to a process for labeling or decorating a plastic object while the object is being formed in a mold. In this process, a label or applique is placed in the open mold and held in the desired position by vacuum ports, electrostatic attraction, or other appropriate means. The mold closes and the molten plastic resin is extruded or injected, or introduced by another equivalent method, into the mold, where it conforms to the shape of the object. The hot plastic envelops the label, making it an integral part of the molded object.

“Thermoforming” refers to a method for preparing a shaped, formed, etc., article, layer, element, component, etc., from a thermoplastic sheet, film, etc. In thermoforming, the sheet, film, etc., may be heated to its melting or softening point, stretched over or into a temperature-controlled, single-surface mold and then held against the mold surface until cooled (solidified). The formed article, layer, element, component, etc., may then be trimmed from the thermoformed sheet. The trimmed material may be reground, mixed with virgin plastic, and reprocessed into usable sheet. Thermoforming may include vacuum forming, pressure forming, twin-sheet forming, drape forming, free blowing, simple sheet bending, etc. “Thermoforming” is also used to describe a method that can comprise the sequential or simultaneous heating and forming of a material onto a mold, wherein the material is originally in the form of a film, sheet, layer, or the like, and can then be formed into a desired shape. Once the desired shape has been obtained, the formed article (e.g., a component of an aircraft interior such as a panel) is cooled below its melt or glass transition temperature. Exemplary thermoforming methods can include, but are not limited to, mechanical forming (e.g., matched tool forming), membrane assisted pressure/vacuum forming, membrane assisted pressure/vacuum forming with a plug assist, and the like.

“Extrusion” refers to a method for shaping, molding, forming, etc., a material by forcing, pressing, pushing, etc., the material through a shaping, forming, etc., device having an orifice, slit, etc., for example, a die, etc. Extrusion may be continuous (producing indefinitely long material) or semi-continuous (producing many short pieces, segments, etc.). The term “coextrusion” and similar terms, such as, for example, “coextruded,” refers to refers to the extrusion of multiple layers of material (e.g., polymers) simultaneously. Coextrusion may utilize two or more extruders to melt and deliver a steady volumetric throughput of different molten materials to a single extrusion head which may combine the materials in the desired extruded shape.

In an embodiment, provided is a monolithic manually operated surgical device comprising: a proximal control means portion for manually operating the device; and a distal operative means portion operably coupled to the proximal control means, wherein the distal operative means portion is operably coupled to the proximal control means and is comprised of a see-through material that is different than the material of the proximal control means portion, wherein (i) the distal operative means portion is transparent, (ii) the distal operative means portion is configured to form a magnifying lens, (iii) the thermoplastic distal operative means is reinforced, (iv) with glass fiber, graphene nanobodies, polyamic acid, carbon nanotubes, a reinforcing solvent, or a combination comprising at least one of the foregoing, and (v) Young's modulus of the proximal control means portion is between 0.1 to 7.0 GPa.

In an embodiment, provided herein is a monolithic, disposable manually operated surgical device comprising: a proximal control means portion for manually operating the device; and a thermoplastic distal operative means portion operably coupled to the proximal control means, wherein the distal operative means portion is comprised of a see-through material that is different than the material of the proximal control means portion, wherein (vi), the see-through thermoplastic operative means portion comprises a first forceps jaw and a second forceps jaw coupled at the proximal ends of the operative means portion to a corresponding first and second distal ends of the proximal control means portion and defining a space between them which can be increased or reduced by operation of the control means, (vii) the first forceps jaw and the second forceps jaw each comprise a protrusion configured to be inserted into a complimentary bore defined in the corresponding first and second distal ends of the control means portion, (viii) the space between the first forceps jaw and the second forceps jaw is 3.0 to 15.0 mm when fully open, (ix) the first forceps jaw and the second forceps jaw each comprises latching means, configured to lock the first forceps jaw to the second forceps jaw, and (x) the device is a suture tying forceps, corneal forceps, iris forceps, eye dressing forceps, epilation forceps, lens holding and folding forceps, artery forceps or ophthalmic forceps and hooks.

In an embodiment, provided is a disposable, monolithic, manually operated surgical device comprising: a proximal control means portion for manually operating the device; and a thermoplastic distal operative means portion operably coupled to the proximal control means, wherein the distal operative means portion is comprised of a see-through material that is different than the material of the proximal control means portion, comprising; (xi) a first and a second opposing lever members pivotally coupled at a pivot point to permit reciprocating movement of the lever members between a closed position and an open position, each lever member comprising: a first distal end adjacent the pivot point, and a thermoplastic handle on a proximal end adjacent the pivot point opposite the distal end, and including a fixed handle loop having an inner loop surface and an outer loop surface, a length of the outer loop surface abutting a corresponding length of outer loop surface of the opposing lever member while in the closed position, wherein (xii) the proximal control means portion comprises the first and second handle ends and the pivot point, (xiii) the see-through distal operative means portion comprises a cutting blade on the first thermoplastic end adjacent the pivot point and a cutting blade on the second thermoplastic end adjacent the pivot point, (xiv) the see-through distal operative means portion comprises a gripping member on the first distal end adjacent the pivot point and a complimentary gripping member on the second distal end adjacent the pivot point, (xv) the distal operative means portion comprises a cutting blade on the first distal end adjacent the pivot point and a cutting blade on the second distal end adjacent the pivot point and the pivot point, and (xvi) the see-through distal operative means portion comprises a gripping member on the first distal end adjacent the pivot point and a complimentary gripping member on the second distal end adjacent the pivot point, and the pivot point.

A more complete understanding of the components, processes, and devices disclosed herein can be obtained by reference to the accompanying drawings. These figures (also referred to herein as “FIG.”) are merely schematic representations based on convenience and the ease of demonstrating the presently disclosed devices, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

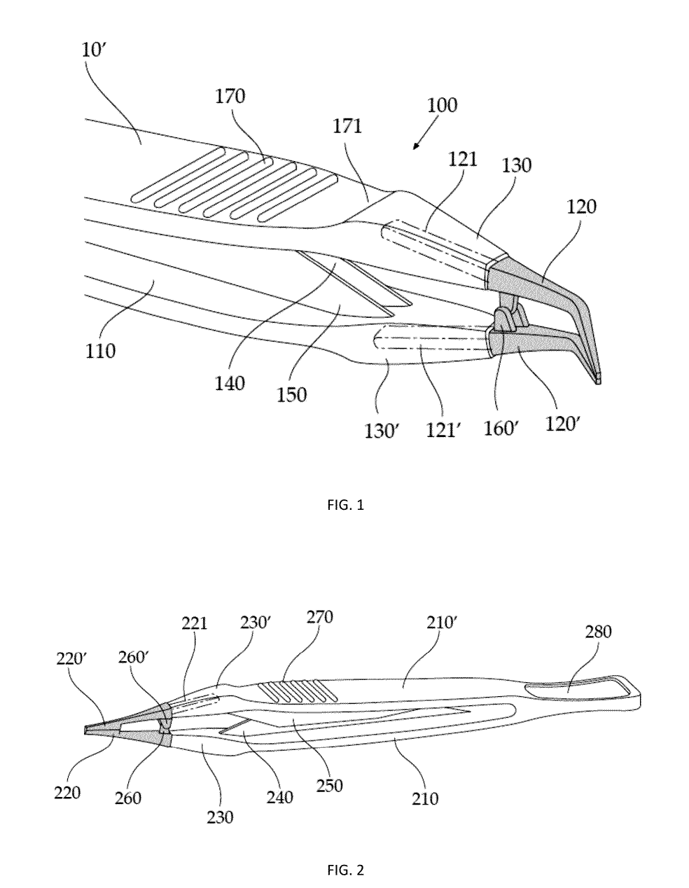

Turning now to FIGS. 1 and 7, showing in FIG. 1 an isometric perspective of a monolithic manually operated iris forceps 100, with a proximal control means portion having a first member 110 and second member 110′ coupled at a proximal end (not shown, see e.g. FIG. 2), terminating in a distal end 130 and 130′ respectively, which can be biased away from each other at an unstressed state, operably coupled to the proximal ends of first member 120 and second member 120′ of the see-through distal operative means portion formed of a material that is different than the material forming the proximal control means portion and can be much stiffer than the thermoplastic material used to form the proximal control portion, for example, with a Young's modulus that is 50 to 70 GPa, reinforced with 0.5-12 mm long fiber glass or metal. As shown in FIG. 1, the forceps can be formed as locking forceps, with a guiding rib 140 extending the length of the first member 110′ of the proximal control means portion, configured to nestingly fit within guiding channel 150 extending the length of the second member 110 of the proximal control means portion, wherein latching means 160, coupled to first member 120 and 160′ coupled to second member 120′ of the distal operating means portion wherein latching means 160 is comprised, for example, of a substantially semi-circular slab that can further comprise protrusions extending laterally from the substantially flat portions of semi circular slab 160, which is configured to nest between the two semi-circular slabs 160′ having complimentary depressions disposed therein. Other locking means, such as interlocking teeth, frictional locking and the like are also envisioned. Additional grip means can be formed on one or both members of the proximal control means portion by adding traverse ribs 170 spanning the width of the first 110′ and/or second 110 members of the proximal control portion and sliding of the fingers. Rising 171 is configured to allow the user to recognize the final access point where the forceps have to be operated. A person skilled in the art, would readily recognize that many alternatives can be made to the shape, dimensions, details and the like, of the see-through devices described herein.

The see-through distal operative means 120 portion is illustrated in FIG. 7, where operative means portion 120 is comprised of an external portion 122 and an internal portion 121, configured to frictionally couple (in other words, be inserted in) to a complimentary recess or bore (not shown) disposed in distal end 130 (e.g., FIG. 1). By 121 or 221 be inside 110 it is reinforcing 130 or 230 Distal operative means can be formed of a see-through, or translucent, or transparent thermoplastic material. Portions 121 and/or 122 can be formed by, for example, injection molding and followed by further processing. As shown in FIG. 7, internal portion 121 can be further modified to include saw-tooth protrusions allowing for monodirectional insertion of the internal portion 121 of the see-through distal operative means portion 120 to the receiving recess or bore in the distal end 130 of member 110. In an embodiment, distal operative means portion 120 can be made of glass (e.g., tempered glass), or an optically clear ceramic, such as alumina, sapphire, ruby, quartz or silica ceramic.

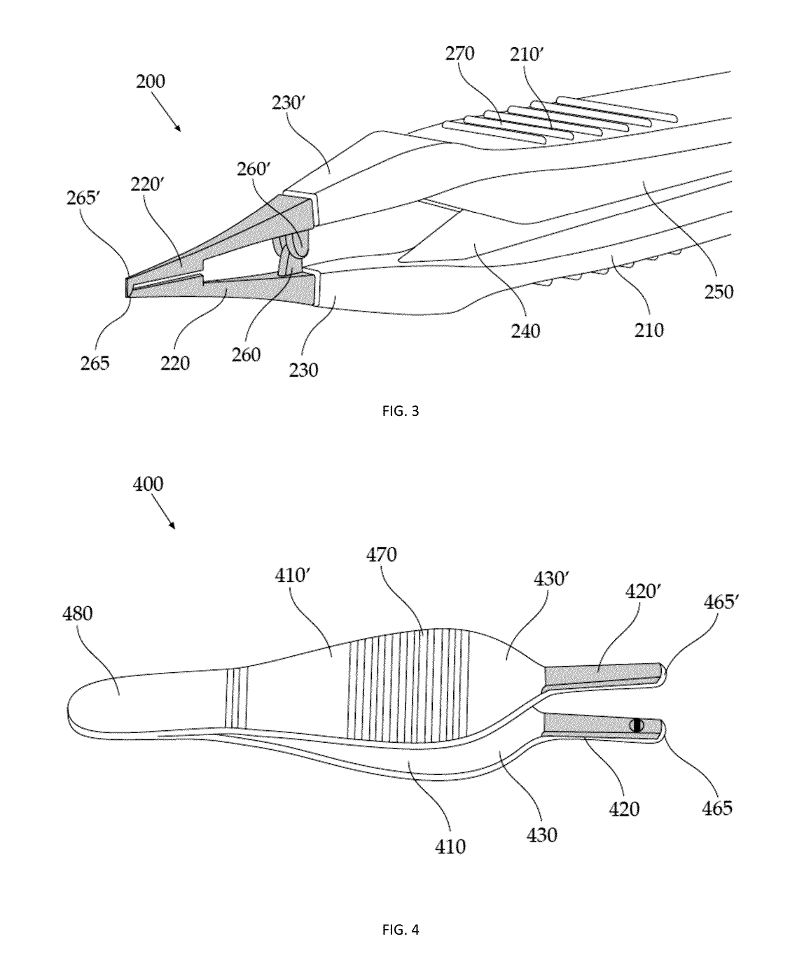

FIGS. 2, 3 and 6 show in FIGS. 2 and 3 an isometric perspective of a disposable monolithic, manually operated suture tying and corneal forceps 200, with a proximal control means portion having a first member 210 and second member 210′ coupled at a proximal end 280 and are biased away from each other at an unstressed state, terminating in the biased distal ends 230 and 230′ respectively, operably coupled to the proximal ends of see-through first member 220 and see-through second member 220′ of the distal operative means portion formed of a material that is different than the material forming the proximal control means portion and can be much stiffer than the thermoplastic material used to form the proximal control portion, for example, with a Young's modulus that is 60 to 280 GPa (e.g., when made of optically transparent ceramic), the distal operative means portion can also be reinforced with grapheme or carbon nanotubes or both. In addition the see-through distal operative can be formed in an angel of a magnifying lens, magnifying the image of a target area bening manipulated by the user.

As shown in FIG. 2, the suture tying and corneal forceps can be formed as locking forceps, with a guiding rib 240 extending the length of the underside of first member 210′ of the proximal control means portion, configured to align and nestingly fit within guiding channel 250 extending the length of the second member 210, when closing the forceps, guiding channel 250 and guiding rib 240 begin guiding in a general rough guiding way followed by moving the “control” to the fine-tuned guiding with latching means 260 and 260′ for assuring closing wherein latching means 260, coupled to first member 220 and 260′ coupled to second member 220′ of the distal operative means portion wherein latching means 260 is comprised of a substantially semi-circular slab that can further comprise protrusions extending laterally from the substantially flat portions of semi circular slab 260, which is configured to nest between the two semi-circular slabs 260′ having complimentary depressions disposed therein. Other locking means, such as interlocking teeth, frictional locking and the like are also contemplated. Additional grip can be formed on one or both members of the proximal control means portion by adding traverse ribs 270 spanning the width of the first 210′ or second 210 members of the reusable proximal control portion. In addition, latching means 260 can be disposed on proximal control means member 210, with complimentary latching means 260′ disposed on proximal control means member 210.

FIG. 3 shows the suture tying and corneal forceps 200, with a proximal control means portion having a first member 210 and second member 210′ coupled at a proximal end 280 and are biased away from each other at an unstressed state, terminating in a distal end 230 and 230′ respectively, operably coupled to the proximal ends of first member 220 and second member 220′ of the disposable thermoplastic distal operative means portion. As shown and described above, forceps jaws 220 and 220′ can have serrated tips (e.g., mouse's teeth). Arrangements of the teeth can be 1×2 (two teeth on one side meshing with a single tooth on the other), 7×7 and 9×9. In an embodiment, teeth 265 and 265′, which may be rat-teeth tip, can be formed of another thermoplastic material that is different than the thermoplastic material forming first member 220 and second member 220′ of the disposable thermoplastic distal operative means portion, and may be also metal.

FIG. 6, shows see-through distal operative means 220 portion, where operative means portion 220 is comprised of an external portion 222 and an internal portion 221, configured to frictionally couple (in other words, be inserted in) to a complimentary recess or bore (not shown) disposed in distal end 230 (e.g., FIG. 3). Distal operative means can be formed of a see-through, or translucent, or transparent thermoplastic material. Portions 221 and/or 222 can be formed by, for example, injection molding and followed by further processing. As shown in FIG. 6, internal portion 221 can be further modified to include saw-tooth protrusions allowing for monodirectional insertion of the internal portion 221 of the see-through distal operative means portion 220 to the receiving recess or bore in the distal end 230 of member 210. In an embodiment, distal operative means portion 220 can be made of glass (e.g., tempered glass), or an optically clear ceramic, such as alumina, sapphire, ruby, quartz or silica ceramic. As shown in FIG. 6, external member 222 having a gripping portion 224 may further comprise a rat tooth tip configuration, with a single tooth 265 on first member 220, nestingly fitting in between two teeth 265′ (not shown) on the second member 220′.

Turning now to FIG. 4, showing a disposable Adson (e.g. tissue grasping) forceps device 400, with a proximal control means portion having a first member 410 and second member 410′ coupled at a proximal end 480 and are biased away from each other at an unstressed state, terminating in a distal end 430 and 430′ respectively, operably coupled to the proximal ends of first see-through member 420 and second see-through member 420′ of the distal operative means portion formed of a material that is different than the material used to form the proximal control means portion and be much stiffer than the thermoplastic material used to form the proximal control portion, for example, with a Young's modulus that is 35 to 225 GPa, where, for example, first distal operative member 420 and second distal operative member 420′ form a magnifying lens. As shown in FIG. 4, first member 420 and second member 420′ of the distal operative means portion may further comprise a rat tooth tip configuration, with a single tooth 465 on first member 420, fitting in between two teeth 465′ on the second member 420′. In an embodiment, teeth 465 and 465′ may be formed of another thermoplastic material that is different than the thermoplastic material forming first member 420 and second member 420′ of the disposable thermoplastic distal operative means portion, and may be also metal.

Turning now to FIG. 5, showing an (Mosquito) hemostat forceps device 500, with a first 510 and a second 510′ opposing lever members pivotally coupled at a pivot point 525 to permit reciprocating movement of the lever members 510, 510′ between a closed position and an open position, each lever member comprising: a first distal end 530, 530′ adjacent pivot point 525, and a handle on a proximal end adjacent pivot point 525 opposite the distal end, and including a fixed handle loop 575, 575′ having an inner loop surface and an outer loop surface. Absent interlocking teeth 560, 560′ a length of the outer loop surface 575 abutting a corresponding length of outer loop surface 575′ of the opposing lever member while in the closed position. As shown in FIG. 5, latching means 560, 560′ comprise a series of interlocking teeth located on a rail extending from outer loop surface of loops 575, 575′. As the handle members 510, 510′ of the forceps 500 comprising finger loops 575, 575′ are closed, the teeth 560, 560′ on first member 510, engage the teeth on second member 510′ and keep the see-through forceps jaws' 520, 520′ grasping surfaces from separating. As shown in FIG. 5, distal ends 530, 530′ terminate in a circle defining an aperture that can be configured to operably couple to pivot point 525, for example, by compression fitting or by any means for attachment allowing for the reciprocating movement of levers 510, 510′. See-through, or translucent or transparent distal operative means portion comprises a gripping member on the first end adjacent the pivot point 520 and a complimentary gripping member on the second end adjacent the pivot point 520′. As shown in FIG. 5, first member 520 and second member 520′ of see-through distal operative means portion are operably coupled to pivot point 525, ensuring synchronized reciprocating movement with the proximal control means portion, which, in certain embodiment can be made of metal, for example.

In addition, distal ends 530, 530′ made of a material that is different than the material of proximal control means portion and can be formed of a transparent material forming a magnifying lens, terminate in an elongated portion extending beyond the circle defining an aperture that can be configured to operably couple to pivot point 525, with distal operative means portion comprising a disposable gripping member on the first thermoplastic end adjacent the pivot point 525 and a complimentary disposable gripping member on the second end 520′ adjacent the pivot point 525, each can comprise a protrusion (not shown, see e.g. 121 on FIG. 6), configured to frictionally fit within a bore (not shown) disposed within distal ends 530, 530′ Likewise, the first end of gripping member 520 adjacent the pivot point 525 and a complimentary gripping member on the second thermoplastic end 520′ adjacent the pivot point 525, each can be a blade.

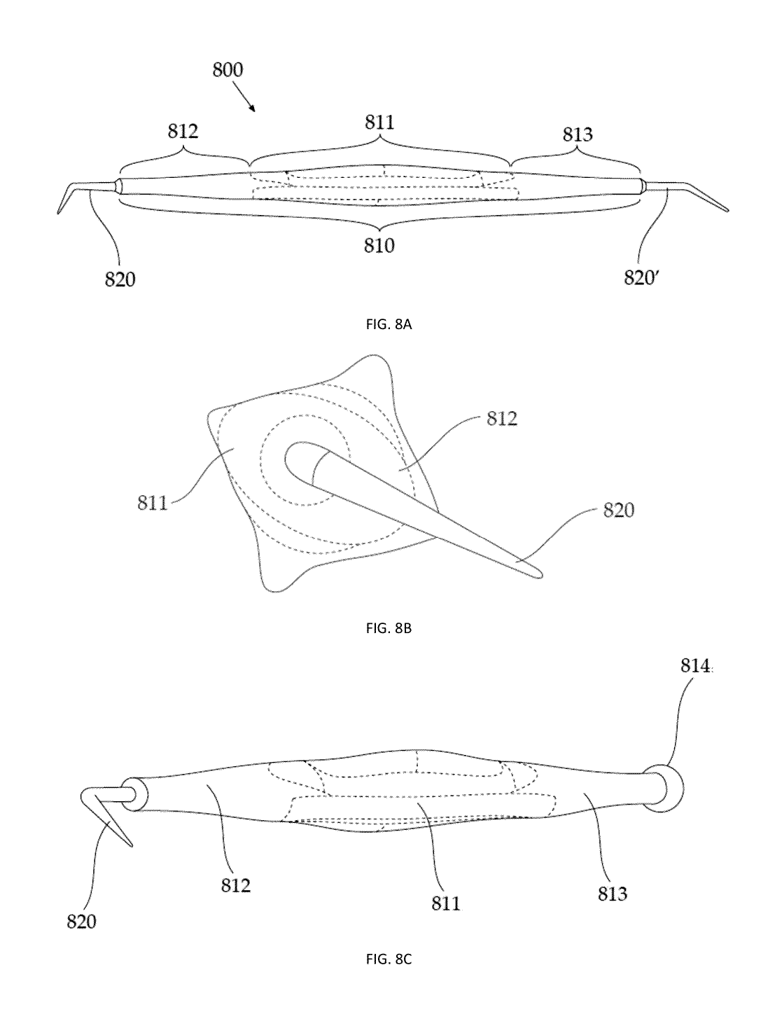

FIG. 8 shows a monolithic, disposable manipulating ophthalmic hook and needle, comprising control means portion 810 coupled to distal operative means 820, 820′ formed from see-through material that is different than the material forming control means portion 810. In an embodiment, forming operative means portion 820 of a different material, reduce the costs and process complexity of disposable manipulating needle 800 and allows separating the process used to form each portion such that the distal operative means portion (e.g., loop manipulator, paddle manipulator, Y-shaped manipulator and the like) to very tight tolerances of +/−1 to 10 μm. The skilled artisan would recognize that these tolerances are neither necessary nor attainable in certain processing methods of the proximal control means portion of the devices described herein. In an embodiment, control means portion 810 is comprised of central section 811 having a non-circular cross section, disposed between two circular cross sections 812, 813 that can be tapered. The non-circular central section 811 of central control means portion 810 can have various cross sections, for example, polygonal, e.g., triangular, or square, or cross-shapes (e.g. a 4-6 lobe torx) and many others that can facilitate roll of the device around its longitudinal axis. As shown in FIGS. 8B and 8C, device 800 can have one or two operative means portions 820 disposed at the edges of control means portion 810. When only one distal see-through operative means portion is coupled to the control means portion 810, the opposite end may terminate in a non-operative portion 814 coupled to control means portion 810.

In addition, distal operative means may require some flexibility and flexure resistance (bending without failure). Accordingly, distal operative means portion 820 (as can all distal operative means portions described herein e.g, blades) can be formed of materials having Young's modulus of between 35 to 280 GPa, with Poisson ratio of between 0.24 to 0.45. The term “flexure-resistant” refers to an element like the manipulating ophthalmic hook and needle which will support a bending moment, in contrast to an element which will support only axial (e.g., compressive) forces. Likewise, as used herein, “flexure resistance” is a means of expressing the flexibility of a material or article such as the distal operative means portion on the devices described herein.

All ranges disclosed herein are inclusive of the endpoints, and the endpoints are independently combinable with each other. “Combination” is inclusive of blends, mixtures, alloys, reaction products, and the like. Furthermore, the terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The term “coupled”, including its various forms such as “operably coupling”, “coupling” or “couplable”, refers to and comprises any direct or indirect, structural coupling, connection or attachment, or adaptation or capability for such a direct or indirect structural or operational coupling, connection or attachment, including integrally formed components and components which are coupled via or through another component or by the forming process. Indirect coupling may involve coupling through an intermediary member or adhesive, or abutting and otherwise resting against, whether frictionally or by separate means without any physical connection. The term “ductile” used herein in accordance with common usage in the art to refer to materials that exhibit significant elongation before break and/or shear yielding in response to an applied force or load during a tensile exposure. In other words, the term “ductile” refers to materials capable of undergoing substantial deformation, e.g., during processing without breaking.

While particular embodiments have been described, alternatives, modifications, variations, improvements, and substantial equivalents that are or may be presently unforeseen may arise to applicants or others skilled in the art. Accordingly, the appended claims as filed and as they may be amended, are intended to embrace all such alternatives, modifications variations, improvements, and substantial equivalents.

I claim:

1. A manually operated surgical device comprising:
a. a proximal control means portion for manually operating the device; and
b. a see-through distal operative means portion operably coupled to the proximal control means, wherein the distal operative means portion is formed of a material that is different than the material forming the proximal control means portion.

2. The device of claim 1, wherein the distal operative means portion is transparent.

3. The device of claim 2, wherein the distal operative means portion is configured to form a magnifying lens.

4. The device of claim 1, wherein the distal operative means is reinforced.

5. The device of claim 4, wherein the thermoplastic distal operative means is reinforced with glass fiber, metal fibers or particles, graphene nanobodies, polyamic acid, carbon nanotubes, a reinforcing solvent, or a combination comprising at least one of the foregoing.

6. The device of claim 1, wherein the thermoplastic material forming the see-through distal operative means is polybutylene terephthalate (PBT); acrylonitrile-butadiene-styrene (ABS); polycarbonate; polycarbonate/PBT blends; polycarbonate/ABS blends; copolycarbonate-polyesters; acrylic-styrene-acrylonitrile (ASA); acrylonitrile-(ethylene-polypropylene diamine modified)-styrene (AES); phenylene ether resins; blends of polyphenylene ether/polyamide; polyamides; phenylene sulfide resins; polyvinyl chloride PVC; high impact polystyrene (HIPS); low/high density polyethylene (L/HDPE); polypropylene (PP), expanded polypropylene (EPP); Polyphthalamide (PPA); or a miscible combination comprising at least one of the foregoing.

7. The device of claim 1, wherein the thermoplastic material forming the proximal control means is polybutylene terephthalate (PBT); acrylonitrile-butadiene-styrene (ABS); polycarbonate (PC); polycarbonate/PBT blends; polycarbonate/ABS blends; copolycarbonate-polyesters; acrylic-styrene-acrylonitrile (ASA); acrylonitrile-(ethylene-polypropylene diamine modified)-styrene (AES); phenylene ether resins; blends of polyphenylene ether/polyamide; polyamides; phenylene sulfide resins; polyvinyl chloride PVC; high impact polystyrene (HIPS); low/high density polyethylene (L/HDPE); polypropylene (PP), expanded polypropylene (EPP); Polyphthalamide (PPA); or a combination comprising at least one of the foregoing.

8. The device of claim 1, wherein the distal operative means portion is formed of glass, optically transparent ceramic or a see-through combination comprising one of the foregoing.

9. The device of claim 1, wherein the operative means portion comprising a first forceps jaw and a second forceps jaw operably coupled at the proximal ends of the operative means portion to a corresponding first and second distal ends of the proximal control means portion and defining a space between them which can be increased or reduced by operation of the control means.

10. The device of claim 9, wherein the first forceps jaw and the second forceps jaw each comprise a protrusion configured to be inserted into a complimentary bore defined in the corresponding first and second distal ends of the control means portion.

11. The device of claim 9, wherein the first forceps jaw and the second forceps jaw each comprises latching means for releasably locking the forceps jaws together configured to lock the first forceps jaw to the second forceps jaw.

12. The device of claim 9, wherein the device is a suture tying forceps, corneal forceps, iris forceps, eye dressing forceps, epilation forceps, lens holding and folding forceps, artery forceps, scissors, Hemostats, Adson forceps, DeBakey forceps, Neuro forceps, bayonet forceps, jewelers forceps, smooth pickups, toothed pickups, serrated tweezers, clamp forceps, lockable crossed tipped Pozzi/Tenaculum Forceps, ophthalmic manipulator, utrata forceps.

13. The device of claim 1, comprising a first and a second opposing lever members pivotally coupled at a pivot point to permit reciprocating movement of the lever members between a closed position and an open position, each lever member comprising: a first distal end adjacent the pivot point, and a thermoplastic or metal handle on a proximal end adjacent the pivot point opposite the first end, and including a fixed handle loop having an inner loop surface and an outer loop surface, a length of the outer loop surface abutting a corresponding length of outer loop surface of the opposing lever member while in the closed position.

14. The device of claim 13, wherein the proximal control means portion comprises the first and second proximal handle ends and the pivot point.

15. The device of claim 13, wherein the distal operative means portion comprises a see-through cutting blade on the first thermoplastic end adjacent the pivot point and a see-through cutting blade on the second thermoplastic or metal end adjacent the pivot point.

16. The device of claim 13, wherein the see-through distal operative means portion comprises a gripping member on the first thermoplastic or metal end adjacent the pivot point and a complimentary gripping member on the second thermoplastic or metal end adjacent the pivot point.

17. The device of claim 13, wherein the see-through distal operative means portion comprises a cutting blade on the first thermoplastic or metal end adjacent the pivot point and a cutting blade on the second thermoplastic end adjacent the pivot point and the pivot point.

18. The device of claim 13, wherein the see-through distal operative means portion comprises a gripping member on the first proximal end adjacent the pivot point and a complimentary gripping member on the second proximal end adjacent the pivot point, and the pivot point.

19. The device of claim 1, where the see-through distal operative means comprises a single manipulating needle.

20. The device of claim 19, wherein the manipulating needle comprises glass, optically transparent ceramic, thermoplastic material or a combination comprising one of the foregoing.
